(19) United States
(12) Patent Application Publication
(10) Pub. No.: US 2006/0251598 A1
(43) Pub. Date: Nov. 9, 2006

(54) System for improved percutaneous absorption of skin benefiting agents

(76) Inventors: Jose E. Ramirez, Trumbull, CT (US); Austin McNamara, Villa Park, CA (US); Judy Hattendorf, Marina Del Ray, CA (US); Steve Goldner, Farmington Hills, MI (US)

Correspondence Address: CARTER, DELUCA, FARRELL & SCHMIDT, LLP, 445 BROAD HOLLOW ROAD, SUITE 225, MELVILLE, NY, 11747, US

(21) Appl. No.: 11/291,223

(22) Filed: Dec. 1, 2005

Related U.S. Application Data
(60) Provisional application No. 60/632,499, filed on Dec. 2, 2004.

(51) Int. Cl. A61K 8/00

(52) U.S. Cl. 424/70.1; 510/130

(57) ABSTRACT

Stable topical compositions containing active ingredients that are prone to oxidation, and methods for improving the percutaneous absorption of an active ingredient are provided.

### CROSS REFERENCE TO RELATED APPLICATION

This application claims priority benefit of U.S. Provisional Application No. 60/632,499 filed Dec. 2, 2004 herein incorporated by reference in its entirety.

### BACKGROUND

1. Technical Field

The present disclosure relates to application of active ingredients and, more specifically to a treatment regimen that improves percutaneous absorption of an active ingredient.

2. Background of the Invention

Many compounds have been applied topically to provide a benefit to the skin of a user. The efficacy of such compounds frequently depends on percutaneous absorption of the compound. An increase in percutaneous absorption would be desirable since less of the active compound would be needed to achieve a desired result. Since the application of active compounds frequently may result in unwanted side effects, an increase in percutaneous absorption would also be desirable since the application of less of the active compound would likely result in a corresponding decrease in unwanted side effects.

Accordingly, it would be desirable to provide more effective percutaneous absorption of active ingredients that are applied topically to the skin of a user.

### SUMMARY

The skin treatment system in accordance with this disclosure includes the sequential application to the skin of a user of a cleanser; a toner; and a composition containing an active ingredient. The skin treatment system further includes the sequential application to the skin of a user of a cleanser; a toner; and a pre-mix containing a blending composition combined with an active ingredient. The present methods can result in an increase in the presence of an active ingredient in the two levels of the skin (i.e., the epidermis, and dermis), compared to application of the active ingredient alone or the pre-mix alone.

### DETAILED DESCRIPTION OF PREFERRED EMBODIMENTS

A treatment regimen in accordance with this disclosure includes the sequential use of three products; namely, a cleanser, a toner and active ingredient composition. The active ingredient composition can be active ingredient alone, any pharmaceutically acceptable composition containing an active ingredient, or a mixture of a blending composition and an active ingredient, or combinations of thereof.

The cleanser can be any non-soap cleanser. Suitable cleansers are commercially available and typically include a combination of anionic, cationic, amphoteric and/or non-ionic surfactants in an aqueous vehicle. The cleanser advantageously can include a combination of compounds to compensate for the well known fact that cleansing agents, by their very nature, are not always well tolerated by the skin. The oil-removal feature of a cleanser can result in drying of the skin, and even skin irritation. By incorporating various protective agents in the cleanser process the cleanser overcomes shortcomings found in many alternative products. Thus, in one particularly useful embodiment the cleanser is a foaming gel cleanser that contains water, sodium lauroyl oat amino acids, cocamidopropyl betaine, sodium laureth sulfate, aloe barbadensis leaf juice, medicago sative (alfalfa) extract, borago officinalis extract, chamomilla recutita (matricaria) extract, sodium chloride, xanthan gum, saponins, phenoxyethanol, methylparaben, propylparaben, ethylparaben, butylparaben, fragrance, and color. This cleanser frees the skin of pollutants without damaging the skin's own natural moisture content. In embodiments, one suitable foaming gel cleanser is Obagi Nu-Derm® foaming gel available from OMP, Inc. of Long Beach, Calif.

Another embodiment uses the commercially available Obagi Nu-Derm® gentle cleanser available from OMP, Inc. of Long Beach, Calif. The Nu-Derm° gentle cleanser contains a combination of water, cocamidopropyl betaine, sodium lauroyl oat amino acids, sodium laureth sulfate, glycerin, aloe barbadensis gel, glycerth-7, apricot triethanolamine, sage extract, borage extract, phenoxythanol, methylparaben, propylparaben, ethylparaben, butylparaben, saponins, fragrance, and colorant.

The toner hydrates and tones the user's skin, while reducing the pH. It prepares the skin for the application of the next step in the regimen. The toner typically contains Hamamelis Virginiana (witch hazel), or some other astringent that tightens and constricts body tissues, as well as provides some secondary cleansing effects. To ameliorate the potentially harsh or drying effects of witch hazel, the following particular combination of ingredients has been developed through testing to produce an astringent that is effective and yet, does not damage or dry out the skin: water, aloe barbadensis leaf juice, hamamelis virginiana (witch hazel) distillate, potassium alum, sodium PCA, panthenol, dmdm hydantoin, polysorbate 80, allantoin, salvia, officianalix (sage) leaf extract, borago officinalis extract, calendula officinalis flower extract, saponins, fragrance and color. One suitable toner is commercially available as Obagi Nu-Derm® toner available from OMP, Inc. of Long Beach, Calif.

The blending composition is designed to be blended with an active ingredient to be applied to the skin. A particularly useful blending composition contains an active ingredient in a base composition of water, glycerin, cetyl alcohol, PPG-2 myristyl ether propionate, sodium lauryl sulfate, TEA-salicylate, lactic acid, phenyl trimethicone, tocopheryl acetate, sodium metabisulfite, ascorbic acid, methylparaben, saponins, disodium EDTA, BHT and propylparaben. One suitable blender is commercially available as Obagi Nu-Derm® blender available from OMP, Inc. of Long Beach, Calif.

Suitable blending compositions may also be made in accordance with the ingredients identified in Table 1.

TABLE 1

| | % of total |
|---|---|
| Compound | composition |
| Purified water | 70-85 |
| Preservatives | .01-1.5 |
| Chelating Agent | .01-0.5 |
| Humectants | 1-10 |
| Anionic | .01-5 |
| Surfactants | |
| Nonionic | .01-5 |
| Surfactants | |
| Organic Acid | .01-5 |
| C12-C18 Alkyl | 2-50 |
| Alcohols | |
| Antioxidants | .01-10 |
| Reducing Agents | .01-5 |
| Emollient | 1-10 |
| Drug ingredient | 0.0-10% |

Suitable preservatives for use in the blending composition further include: benzoic acid, benzyl alcohol, butylparaben, diazolidinyl urea, 2,3-Imidazolidinedione, isopropylparaben, isobutylparaben, methylparaben, propylparaben, sodium butylparaben, sorbic acid, or combinations of these preservatives.

Suitable chelating agents for use in the blending composition further include: citric acid, edetate disodium, ethylenediaminetetraacetic acid, etidronic acid sodium dihydroxyethylglycinate, nitrilotriacetic acid, and combinations of these agents.

Suitable emulsifiers for use in the blending composition further include: cetearyl alcohol, ethoxylated fatty alcohols, PEG-1000 monocetyl ether, alkyl trimethyl ammonium bromide, polyol ester glycerol monostearate, potassium stearate, sodium lauryl sulfate, sodium cetearyl sulfate, saponins, and combinations of these agents.

Suitable humectants for use in the blending composition further include: glycerin, diglycerin, triglycerin, polyglycerin, polypropylene glycol, polyethylene glycol, ethylene glycol, diethylene glycol, triethylene glycol, propylene glycol, dipropylene glycol, hexylene glycol, 1,3-butylene glycol, 1,4-butylene glycol, ethylene glycol monoalkyl ether, diethylene glycol monoalkyl ether, glucose, maltose, sucrose, lactose, xylitose, xylitol, sorbitol, mannitol, maltitol, panthenol, pentaerythritol, hyaluronic acid, and combinations of these humectants.

Suitable pH adjusters for use in the blending composition further include: citric acid, phosphoric acid, lactic acid, glycolic acid, and combinations of these pH adjusters.

Suitable antioxidants for use in the blending composition further include: ascorbyl palmitate, 2,6 ditertiarybutyl-4-methyl phenol, butylated hydroxyanisole, tocopherol, tocopheryl acetate, propyl gallate, and combinations of these antioxidants.

Suitable emollients for use in the blending composition further include: cetyl alcohol, stearyl alcohol, liquid hydrocarbon oil, liquid natural oil, liquid fatty alcohol, liquid fatty acid, liquid fatty acid ester, liquid silicone oil, paste wax, and combinations of these emollients.

Suitable reducing agents for use in the blending composition further include: ascorbic acid, propyl gallate, sodium metabisulfite, and combinations of these reducing agents.

Non-limiting examples of active ingredients which may be applied to the skin in a pharmaceutically acceptable composition, or mixed into a blending composition are listed below. The active ingredients are categorized in various classes however this classification is not intended to limit the active ingredients in any way to only to those actives belonging to the categories herein mentioned.

Sunscreen Actives

A wide variety of sunscreen actives are useful herein. The exact amount and type of sunscreen that is used depends on the level of photoprotection that is desired. Generally any agent offering protection against ultraviolet radiation by absorbing, scattering or reflecting the ultraviolet radiation may be used herein. The sunscreen agents used herein may offer protection against one or more of the following forms of sunlight radiation UVA, UVB, UVC, visible light and infrared radiation. Generally the sunprotection factor (SPF) in the final formulation varies between 2 and 30, although products with SPFs up to 100 may be formulated. The sunscreen used herein may offer chemical or physical photoprotection.

Sunscreens which may be used in accordance with the present disclosure include those selected from the group comprising amino benzoic acid and derivatives, such as para-amino benzoic acid (PABA), glyceryl-PABA (Lisadimate), Padimate O, Roxadimate; anthrinalates, including methylanthrynilate; benzophenones, including dioxybenzone, oxybenzone and sulisobenzone, 3-benzophenone (Uvinul M40) 4-N,N-dimethylaminobenzoic acid ester with 2,4-dihydroxybenzophenone; camphor derivatives including 3-(4-methylbenzylidene) camphor, 3-benzylidene camphor; cinnamates including DEA-p-methoxycinnamate, ethyl-hexyl p-methoxy cinnamate, octocrylene, octyl methoxy cinnamate (Parasol MCX); dibenzoyl methanes including butylmethoxydibenzoylmethane (Parsol 1789), salicylates including, homomenthyl salicylate, octyl salicylate, trolamine methyl salicylate; metal oxides including titanium dioxide, zinc oxide and iron oxide; 2-phenylbenzimidazole-5-sulfonic acid; 4,4-methoxy-t-butyidibenzoylmethane; and mixtures thereof.

Anti-wrinkle and Anti-aging Actives

Anti-wrinkle and anti-aging actives suitable for use in accordance with the present disclosure include without limitation hydroxy acids including C₂-C₃₀ alpha-hydroxy acids such as glycolic acid, lactic acid, 2-hydroxy butanoic acid, malic acid, citric acid tartaric acid, alpha-hydroxyethanoic acid, hydroxycaprylic acid and the like; beta hydroxy acids including salicylic acid and polyhydroxy acids including gluconolactone (G4); and mixtures of these acids. Further anti-wrinkle agents include retinoic acid, gamma-linolenic acid; fruit acids, sugar cane extract and glycomer in cross-linked alpha nutrium; and mixtures thereof. Skin peel agents for example phenol, phytic acid and acetic acid may also be used in accordance with the present disclosure. In embodiments, salicylic acid, lactic acid and glycolic acid are suitable for use herein.

Whitening and Bleaching Actives

Whitening and bleaching agents include hydroquinone, kojic acid; lactic acid; ascorbyl acid and derivatives such as magnesium ascorbyl phosphate; arbutin; and licorice root.

Sunless Tanning Actives

Sunless tanning actives include dihydroxyacetone (DHA); glyceryl aldehyde; tyrosine and tyrosine derivatives such as malyltyrosine, tyrosine glucosinate, and ethyl tyrosine; phospho-DOPA, indoles and derivatives; and mixtures thereof.

Antimicrobial Actives

Antimicrobials suitable for use in accordance with the present disclosure include all antibiotics, antimicrobial agents and antimicrobial peptides. Antibiotics that may be used include inter alia dermatologically acceptable salts of tetracyclin and tetracycline derivatives, gentamycin, kanamycin, streptomycin, neomycin, capreomycin, lineomycin, paromomycin, tobramycin, erythromycin, triclosan, octopirox, parachlorometa xylenol nystatin, tolnaftate, miconazole hydrochloride, chlorhexidine gluconate, chlorhexidine hydrochloride, methanamine hippurate, methanamine mandelate, minocycline hydrochloride, clindamycin, cleocin, b-lactam derivatives such as aminopenicillin and mixtures thereof. In embodiments, chlorhexidine gluconate and triclosan are suitable for use herein.

Anti microbial agents that may be used in accordance with the present disclosure include for example benzoyl peroxide and salicylic acid.

Antimicrobial peptides useful herein are for example magainin, nicin and cecropin.

Anti-acne Actives

Anti-acne actives suitable for use in accordance with the present disclosure include without limitation tretinoin, keratolytic agents including lactic acid, pyruvic acid, salicylic acids, urea and N-acetylcysteine; retinoids, and retinoid analogs such as tretinoin, cis and trans retinoic acid, retinol and retinol palmitate, isotretinoin-13-cis-retinoic acid; antibiotics and antimicrobial agents such as tetracycline, erythromycin, minocycline, clindamycin, trimethoprim-sulphamethazole and anti-microbial peptides (nicin, for example); steroids, such as hydrocortisone; gamma-linolenic acid and mixtures thereof. Further anti-acne actives that may be used include without limitation benzoyl peroxide; alpha and beta hydroxy acids; sulfacteamide and sulfur and mixtures thereof. In. embodiments, salicylic acid, benzoyl peroxide and retinoids are suitable for use herein.

Anti-psoriasis Actives

Anti-psoriasis actives suitable for use in accordance with the present disclosure include without limitation salicylic acid; mometasone furoate; steroids including corticosteroids such as cortisone and oluxclobetasol propionate; 5-fluorouracil; epinephrine; anthralin; vitamin D3 analogs, such as calcipotriene; methotrexate; masprocol; trimethaxate gluconate; retinoids; cyclosporin; paclitaxel; 5-amino levulinic acid; bergasol; tin-ethyl etio purpurin; benzoporphyrin derivatives; antibodies, such as ABX-IL8 antibody, CD11a monoclonal antibody and ICM3 monoclonal antibody; enzyme inhibitors, including tryptase inhibitor and phospholipase A-2 inhibitors; angiogenesis blocking agents; T-cell blocking agents and mixtures thereof. (h)

Anti Eczema Actives

Anti-eczema actives suitable for use in accordance with the present disclosure include urea; evening primrose oil; plant extracts; hydrocortisone; an immunomodulator; tar combined with fatty acids obtained from banana; and mixtures thereof.

Topical Anesthetic Actives

Topical anesthetic actives suitable for use in accordance with the present disclosure include tetracaine, lidocaine, editocaine, bupivacaine, pramoxine; and mixtures thereof.

Anti-inflammatory Actives

Anti-inflammatory actives suitable for use in accordance with the present disclosure include steroidal actives such as hydrocortisone as well as non-steroidal actives including propionic derivatives; acetic acid derivatives; biphenylcarboxylic acid derivatives; fenamic acid derivatives; and oxicams. Examples of anti-inflammatory actives include without limitation acetaminophen, oxaprozin, pranoprofen, benoxaprofen, bucloxic acid, elocon; and mixtures thereof.

Vitamin Actives

Vitamin actives suitable for use in accordance with the present disclosure include vitamin A and derivatives, including retinoic acid, retinyl aldehyde, retin A, retinyl palmitate, adapalene, and beta-carotene; vitamin B (panthenol, provitamin B5, panthenic acid, vitamin B complex factor); vitamin C (ascorbic acid and salts thereof) and derivatives such as ascorbyl palmitate; vitamin D including calcipotriene (a vitamin D3 analog) vitamin E including its individual constituents alpha-, beta-, gamma-, delta-tocopherol and cotrienols and mixtures thereof and vitamin E derivatives including vitamin E palmitate, vitamin E linolate and vitamin E acetate; vitamin K and derivatives; vitamin Q (ubiquinone) and mixtures thereof.

Protein Actives

One class of actives suitable for use in accordance with the present disclosure are proteins and peptides. In principle, any desired protein or peptide may be produced using this technology and oil bodies comprising these recombinant proteins may be incorporated in the emulsions of the present disclosure. Proteins and peptides which may be used in accordance with the present disclosure include enzymes such as proteases (e.g. bromelain, papain, collagenase, elastase), lipases (e.g. phospholipase C), esterases, glucosidases, exfoliating enzymes; antibodies and antibody derived actives, such monoclonal antibodies, polyclonal antibodies, single chain antibodies and the like; reductases; oxidases; peptide hormones; natural structural skin proteins, such as elastin, collagen, reticulin and the like; growth factors such as platelet derived growth factor (PDGF) and epidermis derived growth factor (EGF); anti-oxidants such as superoxide dismutase, catalase and glutathione; free-radical scavenging proteins; DNA-repair enzymes, for example T4 endonuclease 5 and P53; antimicrobial peptides, such as magainin and cecropin; a milk protein; a silk protein or peptide; and any active fragments, derivatives of these proteins and peptides; and mixtures thereof.

Miscellaneous Active Ingredients

Further active ingredients suitable for use in accordance with the present disclosure include an amino acid and amino acid derivative; an insect repellant; a fungicide (such as ketoconazole); an anti-viral agent (such as acyclovir); an anti-cancer agent; a plant extract; an anti-hemorrhoid compound; an anti-dandruff compound; a hair-growth stimulating compound; a hair loss stimulating compound; a nucleic acid (DNA, RNA and derivatives); an anti-scabies agent (such as permethrin); an anti-wart agent (such as podophyllotoxin); and mixtures thereof.

Pharmaceutically acceptable active ingredient compositions relate to any formulation that contains any active ingredient for use in accordance with the present disclosure. For topical applications, the pharmaceutical compositions may be formulated in a suitable ointment containing the active ingredient suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active ingredients suspended or dissolved in one or more pharmaceutically acceptable carriers. Suitable carriers include, but are not limited to, mineral oil, sorbitan monostearate, polysorbate, cetyl esters wax, cetearyl alcohol, 2-octyldodecanol, benzyl alcohol and water. For example a 20% Vitamin C composition may be formulated in water and other ingredients for promoting stability, which may be topically applied to the skin.

In embodiments, the various compositions can be formulated, manufactured and packaged in accordance with this disclosure in a manner which enables the composition to remain in the package without discoloring. As used herein the term “stable” means that the composition when in a closed container remains within the tolerances and limits set forth in US Pharmacopeia and/or the US FDA guidelines or monographs for compositions containing any particular active ingredient or combination of active ingredients. The entire US Pharmacopeia and collection of US FDA guidelines or monographs for compositions containing any particular active ingredient or combination of active ingredients are too voluminous to present in their entirety herein and thus are instead incorporated in their entirety by this reference. With respect to topical compositions, the tolerances and limits are frequently presented relative to the labeled amount. As one illustrative example, for hydroquinone cream, the acceptable tolerance is not less than 94.0 percent and not more than 106.0 percent of the labeled amount of C₆H₆O₂. As another illustrative example, for tretinoin cream, the acceptable tolerance is not less than 90.0 percent and not more than 130.0 percent of the labeled amount of C₂₀H₂₈O₂. Those skilled in the art will readily be able to identify the tolerances and limits for other compositions containing other active ingredients.

As those skilled in the art will appreciate, the container-liner-closure system used to store the composition will affect the stability of the active ingredient. It should be understood that a composition need not be stable in all containers to be stable in accordance with this disclosure. Stability in at least one type of container is sufficient for a composition to be stable as that term is used herein.

In one particularly useful method in accordance with this disclosure, a cleanser is used to cleanse the area of skin to be treated and the residue wiped off. Cleanser is applied in an amount suitable for cleaning the skin area to be cleaned. Subsequently toner is applied, rubbed in and allowed to dry. Toner is applied in an amount suitable for toning the skin area to be treated. An aqueous solution of active ingredient is prepared, for example 20% vitamin C solution. The solution is then applied to the skin in an amount suitable to obtain a desired effect.

Blending composition may also be mixed with active ingredients in amounts sufficient to provide a benefit to skin. For example, a premix of 100 mg of a commercially available oil-in-water blending composition containing 4% Hydroquinone and 100 mg of tretinoin cream 0.1% tretinoin was prepared by mixing the two components in a 1:1 ratio. The concentration was such that only about 30 mg of pre-mixed hydroquinone/tretinoin would be needed to provide a dosage designed to benefit the skin. Here the active ingredient is tretinoin which is combined with the drug ingredient of the blender, hydroquinone.

In another particularly useful method in accordance with this disclosure, a cleanser is used to cleanse the area of skin to be treated and the residue wiped off. Cleanser is applied in an amount suitable for cleaning the skin area to be cleaned. Subsequently toner is applied, rubbed in and allowed to dry. Toner is applied in an amount suitable for toning the skin area to be treated. A premix of a blending composition and active ingredient is prepared. The pre-mix is then applied to the skin in an amount suitable to obtain the desired effect.

Optionally, in another particularly useful method in accordance with this disclosure, a cleanser is used to cleanse the area of skin to be treated and the residue wiped off. Cleanser is applied in an amount suitable for cleaning the skin area to be cleaned. Subsequently toner is applied, rubbed in and allowed to dry. Toner is applied in an amount suitable for toning the skin area to be treated. An active ingredient is preselected. The active ingredient is then applied to the skin in an amount suitable to obtain the desired effect.

In order that those skilled in the art may be better able to practice the compositions and methods described herein, the following example is given for illustration purposes.

### EXAMPLE 1

### Localization of Tretinoin in Human Skin

An in-vitro study of percutaneous absorption of tretinoin formulations in human cadaver skin was conducted. In this study a tretinoin cream containing 0.1% tretinoin was applied to different cadaver skin samples. The following application protocols were used on the different groups of Franzs Cells:

Treatment A. 15 mg of tretinoin was applied to 1.77 cm² of human cadaver skin samples.

Treatment B. A premix of 100 mg of a commercially available o/w composition containing 4% Hydroquinone and 100 mg of tretinoin cream 0.1% tretinoin was prepared. 30 mg of Pre-Mixed Hydroquinone/Tretinoin was applied to the samples.

Treatment C. A commercial cleanser was used to cleanse the sample area and the residue wiped off. Subsequently a toner was applied, rubbed in and allowed to dry. A premix of 100 mg of a blending composition and 100 mg of tretinoin cream 0.1% tretinoin was prepared. 30 mg of Pre-Mixed Hydroquinone/Tretinoin was applied to the samples.

There were no significant differences in percutaneous absorption of Tretinoin in treatments A, or B. In Treatment C in accordance with the present disclosure where the skin samples were pretreated with a cleanser and toner prior to the application of mixture of blending composition/Tretinoin, results showed significant increases in Tretinoin percutaneous absorption in the different dermal skin layers (stratum corneum, epidermis and dermis) when compared to all the other treatments tested. Therefore a treatment of specifically designed products, e.g., cleanser, toner and blending composition/active ingredient pre-mix works better than the individual products alone. For example, the dermis penetration of treatment C is 3× greater than the dermis penetration of Treatment A.

The results also showed for Treatment A that 71.6% active passed through the skin and 19.2% active passed through the dermis. For treatment B, 66.3% active passed through the skin, and 18% active passed through the dermis. For treatment C, 79.2% active passed through the skin, and 26.8 active passed through the dermis. Thus, where the skin samples were pretreated with a cleanser and toner prior to the application of mixture of blending composition/Tretinoin, results showed significant increases in Tretinoin percutaneous absorption in the different dermal skin layers (dermis, epidermis, stratum corneum, and stratum corneum surface).

Results: Results showing the localization of tretinoin in human skin samples are shown below in Table 2 for treatments A, B and C.

TABLE 2

| | | | STR. | SC |
|---|---|---|---|---|
| TREATMENT | DERMIS | EPIDERMIS | COR. | SURFACE |
| A | 0.02 ± 0.01 | 0.21 ± 0.06 | 0.42 ± 0.16 | 0.34 ± 0.12 |
| n = | 9 | 9 | 9 | 9 |
| B | 0.03 ± 0.01 | 0.14 ± 0.06 | 0.32 ± 0.11 | 0.32 ± 0.14 |
| n = | 9 | 8 | 9 | 9 |
| C | 0.06 ± 0.02 | 0.39 ± 0.13 | 0.51 ± 0.20 | 0.35 ± 0.16 |
| n = | 9 | 9 | 9 | 9 |

### EXAMPLE 2

An in-vitro study of percutaneous absorption of vitamin C formulation in human cadaver skin was conducted. In this study a vitamin C compositions were applied to different cadaver skin samples. The following application protocols were used on the different groups of skin cells:

Treatment A. 20% vitamin C composition was applied to human cadaver skin samples.

Treatment B. Obagi Nu-Derm® cleanser, toner, and 20% vitamin C composition was applied to human cadaver skin samples.

In treatment B where the skin samples were pretreated with a cleanser and toner prior to the application of vitamin C compositions, results showed significant increases in vitamin C percutaneous absorption in skin when compared to Treatment A. Accordingly, skin pretreated with a cleanser and toner showed increased percutaneous absorption of vitamin C active ingredient.

The results of the tests are shown below in Table C.

TABLE C

| | | | % Vitamin C in |
|---|---|---|---|
| Treatment | Product | % Vitamin C | Epidermis |
| A | Vitamin C | 20% | 9.8% |
| | composition | | |
| B | Obagi Nu- | 20% | 15.8% |
| | Derm ® | | |
| | Cleanser + Toner + Vitamin | | |
| | C composition | | |
| | of A | | |

It will be understood that various modifications may be made to the embodiments disclosed herein. Therefore, the above description should not be construed as limiting, but merely as exemplifications. Those skilled in art will envision other modifications within the scope and spirit of the claims appended hereto.

What is claimed is:

1. A method of improving the percutaneous absorption of an active ingredient topically applied to the skin of a user, the method comprising:
applying a cleanser to the skin of a user;
applying a toner to the cleansed skin;
applying an active ingredient to the cleansed, toned skin,
wherein percutaneous absorption of the active ingredient is increased compared to application of the active ingredient to untreated skin.

2. The method of claim 1 wherein the active ingredient is tretinoin.

3. The method of claim 1 wherein the active ingredient is Vitamin C.

4. The method of claim 1 wherein the active ingredient is in a pharmaceutically acceptable composition.

5. The method of claim 4 wherein the composition further comprises a carrier for topical administration.

6. The method of claim 4 wherein the composition is a lotion or cream.

7. A method of improving the percutaneous absorption of an active ingredient topically applied to the skin of a user, the method comprising:
applying a cleanser to the skin of a user;
applying a toner to the cleansed skin;
pre-mixing an active ingredient with a blending composition to form a pre-mix; and
applying the pre-mix to the cleansed, toned skin,
wherein percutaneous absorption of the active ingredient is increased compared to application of the active ingredient or the pre-mix to untreated skin.

8. The method of claim 7 wherein the blending composition comprises: an active ingredient sensitive to oxidation; a preservative; a chelating agent; an emulsifier; a humectant; a pH adjuster; an antioxidant; an emollient; a reducing agent, and water.

9. The method of claim 8 wherein the preservative comprises benzoic acid, benzyl alcohol, butylparaben, diazolidinyl urea, 2,3-Imidazolidinedione, isopropylparaben, isobutylparaben, methylparaben, propylparaben, sodium butylparaben, sorbic acid, or combinations thereof.

10. The method of claim 8 wherein the chelating agent comprises citric acid, edetate disodium, ethylenediaminetetraacetic acid, etidronic acid sodium dihydroxyethylglycinate, nitrilotriacetic acid, or combinations thereof.

11. The method of claim 8 wherein the emulsifier comprises cetearyl alcohol, ethoxylated fatty alcohols, PEG-1000 monocetyl ether, alkyl trimethyl ammonium bromide, polyol ester glycerol monostearate, potassium stearate, sodium lauryl sulfate, sodium cetearyl sulfate, saponins, or combinations thereof.

12. The method of claim 8 wherein the humectant comprises glycerin, diglycerin, triglycerin, polyglycerin, polypropylene glycol, polyethylene glycol, ethylene glycol, diethylene glycol, triethylene glycol, propylene glycol, dipropylene glycol, hexylene glycol, 1,3-butylene glycol, 1,4-butylene glycol, ethylene glycol monoalkyl ether, diethylene glycol monoalkyl ether, glucose, maltose, sucrose, lactose, xylitose, xylitol, sorbitol, mannitol, maltitol, panthenol, pentaerythritol, hyaluronic acid, or combinations thereof.

13. The method of claim 8 wherein the pH adjuster comprises citric acid, phosphoric acid, lactic acid, glycolic acid, or combinations thereof.

14. The method of claim 8 wherein the antioxidant comprises ascorbyl palmitate, 2,6 ditertiarybutyl-4-methyl phenol, butylated hydroxyanisole, tocopherol, tocopheryl acetate, propyl gallate, or combinations thereof.

15. The method of claim 8 wherein the emollient comprises cetyl alcohol, stearyl alcohol, liquid hydrocarbon oil, liquid natural oil, liquid fatty alcohol, liquid fatty acid, liquid fatty acid ester, liquid silicone oil, paste wax, or combinations thereof.

16. The method of claim 8 wherein the reducing agent comprises ascorbic acid, propyl gallate, sodium metabisulfite, or combinations thereof.

17. The method of claim 8 wherein the active ingredient sensitive to oxidation is hydroquinone.

17. The method of claim 7 wherein the active ingredient is tretinoin.

18. The method of claim 7 wherein the premix is a lotion or cream.
